(19) United States
(12) Patent Application Publication
(10) Pub. No.: US 2007/0049756 A1
(43) Pub. Date: Mar. 1, 2007

(54) ALFUZOSIN HYDROCHLORIDE POLYMORPHS

(76) Inventors: Raghupathi Reddy Anumula, Hyderabad 500 072, A.P. (IN); Sampath Alla, Karim Nagar 505 152, A.P. (IN); Lokeswara Rao Madivada, Krishna, A.P. (IN); Goverdhan Gilla, Hyderabad 500 050, A.P. (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/467,951

(22) Filed: Aug. 29, 2006

Related U.S. Application Data
(60) Provisional application No. 60/787,704, filed on Mar. 30, 2006.

(30) Foreign Application Priority Data
Aug. 30, 2005 (IN) 1210/CHE/2005

(51) Int. Cl. C07D 405/02

(52) U.S. Cl. 544/284

(57) ABSTRACT

Alfuzosin hydrochloride crystalline and amorphous polymorphic forms and processes for preparing them.

The present invention relates to crystalline and amorphous forms of alfuzosin hydrochloride and processes for their preparation.

Alfuzosin hydrochloride has the chemical name (R,S)-N-[3-[(4-amino-6,7-dimethoxy-2-quinazolinyl)methylamino]propyl]tetrahydro-2-furancarboxamide hydrochloride, and can be depicted by the structural Formula I.

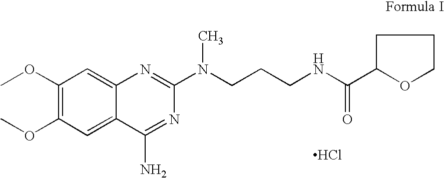

Alfuzosin hydrochloride is known to be an antagonist of α-adrenergic receptors, and is useful as an antihypertensive agent and dysuria agent. It is sold in pharmaceutical products using the UROXATRAL trademark as 10 mg extended release tablets.

U.S. Pat. No. 4,315,007 discloses alfuzosin hydrochloride, a pharmaceutical composition and their use in treating cardio vascular disorders. It also discloses a process for the preparation of alfuzosin hydrochloride (alfuzosin hydrochloride prepared according to this process being hereinafter referred to as “Form I” for convenience).

U.S. Pat. No. 5,545,738 discloses alfuzosin hydrochloride anhydrous, the dihydrate, the trihydrate, and the tetrahydrate.

Regulatory authorities require that efforts be made to identify all polymorphic forms, e.g., crystalline, amorphous, solvated, etc., of new drug substances, since polymorphic forms can vary in their chemical and physical properties. This variation frequently results in bioavailability, stability, and other differences between production lots of formulated pharmaceutical products.

However, the existence, and possible numbers, of polymorphic forms for a given compound cannot be predicted. In addition, there are no “standard” procedures that can be used to prepare polymorphic forms of a substance.

Therefore, there is a need for preparing new solid forms of a drug substance and processes for preparation thereof.

Thus, the present invention is directed toward the development of new solid forms of alfuzosin hydrochloride and processes for preparation thereof.

### SUMMARY OF THE INVENTION

The present invention provides crystalline polymorphic forms of alfuzosin hydrochloride described herein, designated as Form II, Form III and Form IV, which are characterized by their X-ray powder diffraction (XRPD) patterns. In accordance with one aspect, the present invention provides crystalline Forms II, III, and IV of alfuzosin hydrochloride.

The crystalline Form II of alfuzosin hydrochloride has an X-ray powder diffraction pattern that contains peaks at about 8.1, 14.4 and 17.9, ±0.2° 2θ.

The crystalline Form III of alfuzosin hydrochloride has an X-ray powder diffraction pattern that contains peaks at about 7.4 and 10.6, ±0.2° 2θ.

The crystalline Form IV of alfuzosin hydrochloride has an X-ray powder diffraction pattern that contains peaks at about 10.0, 18.3 and 23.2, ±0.2° 2θ.

In accordance with other aspects, the present invention provides a process for preparing the crystalline Forms II, III and IV.

A process for the preparation of crystalline Forms II, III and IV of alfuzosin hydrochloride comprises the steps of:

a) reacting alfuzosin free base with hydrochloric acid in a suitable solvent;

b) optionally adding another solvent; and

c) recovering the desired crystalline form.

In accordance with another aspect the present invention also provides an amorphous form of alfuzosin hydrochloride, which is characterized by its X-ray powder diffraction (XRPD) pattern.

In accordance with one more aspect, the present invention provides a process for preparing the amorphous form. The process for the preparation of amorphous form comprises the steps of:

a) heating alfuzisin hydrochloride comprising water to melt; and

b) cooling the liquid of step a) to generate the amorphous form.

In accordance with yet another aspect, the invention provides pharmaceutical compositions containing one or more pharmaceutically acceptable excipients and a prophylactically or therapeutically effective amount of, individually or as mixtures in any proportions, the crystalline Forms II, III, and IV, and amorphous, alfuzosin hydrochloride.

Further in accordance with yet another aspect, the present invention provides a process for the conversion of alfuzosin hydrochloride Form II to anhydrous alfuzosin hydrochloride Form I comprising heating alfuzosin hydrochloride Form II at a suitable temperature.

Also, the invention provides a method of treating or preventing a patient suffering from cardiovascular disorders by administering the patient a prophylactically or therapeutically effective amount of individually or as mixtures in any proportions of the crystalline Forms II, III, and IV, and amorphous, alfuzosin hydrochloride.

### BRIEF DESCRIPTION OF THE DRAWINGS

FIG. 1 is an X-ray diffraction pattern of the crystalline Form II of alfuzosin hydrochloride prepared according to Example 1.

FIG. 2 is a differential scanning calorimetric analysis curve of the crystalline Form II of alfuzosin hydrochloride prepared according to Example 1.

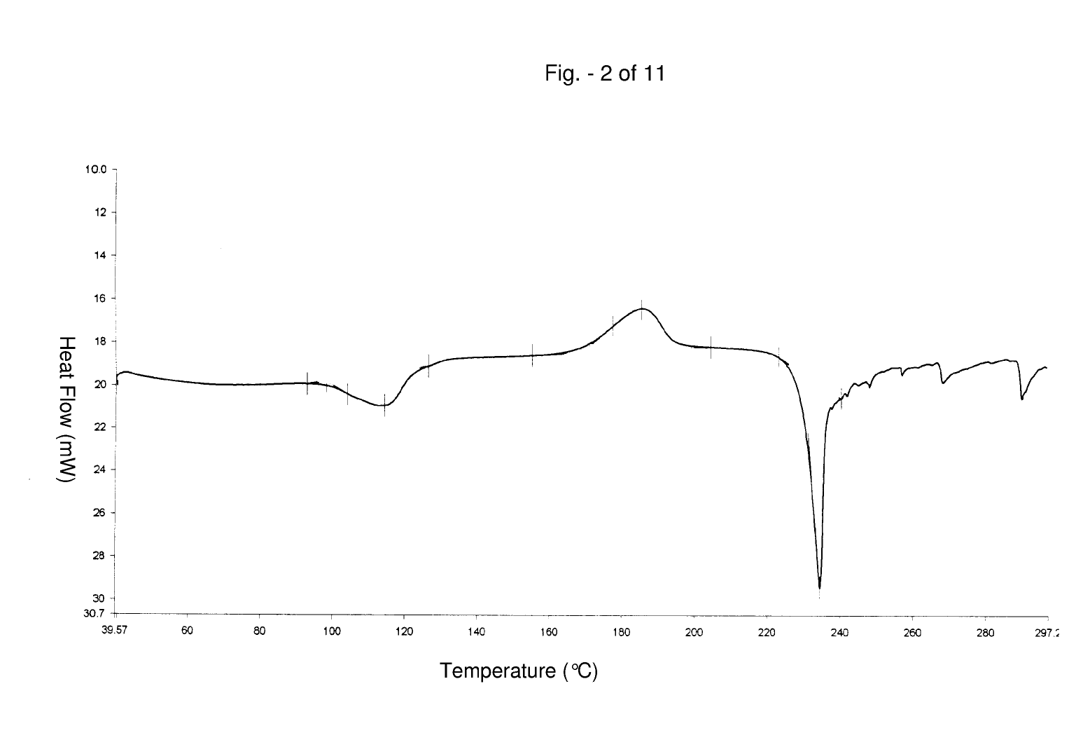

FIG. 3 is a thermogravimetric analysis curve of the crystalline Form II alfuzosin hydrochloride prepared according to Example 1.

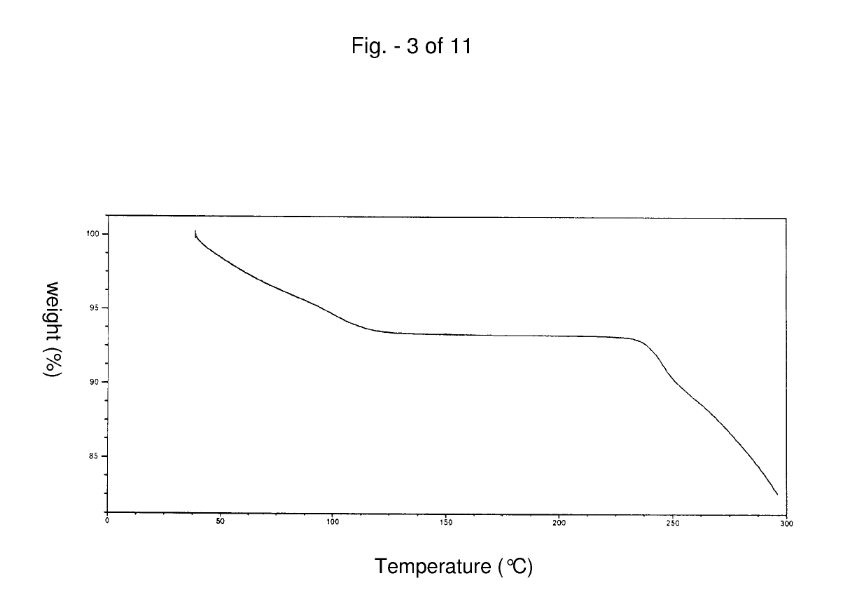

FIG. 4 is an X-ray diffraction pattern of the crystalline Form III of alfuzosin hydrochloride prepared according to Example 3.

FIG. 5 is a differential scanning calorimetric analysis curve of the crystalline Form III of alfuzosin hydrochloride prepared according to Example 3.

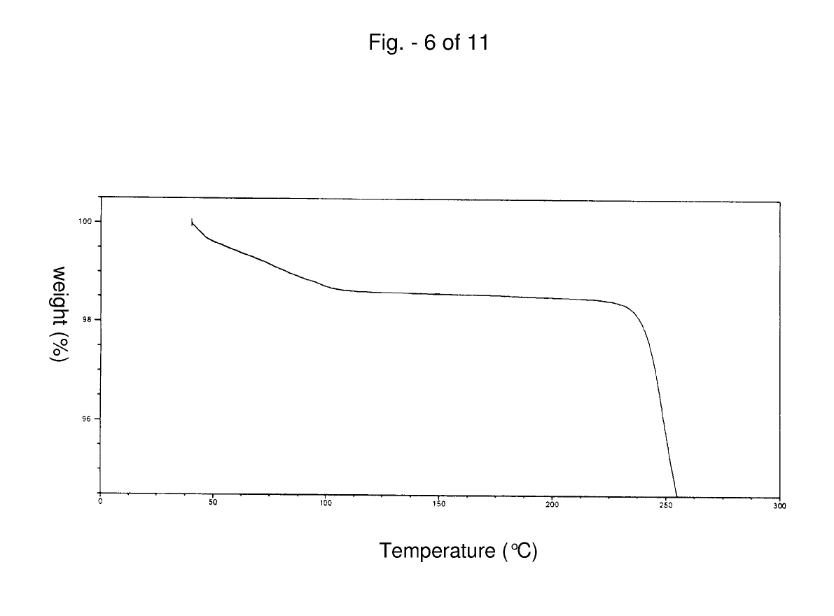

FIG. 6 is a thermogravimetric analysis curve of the crystalline Form III alfuzosin hydrochloride prepared according to Example 3.

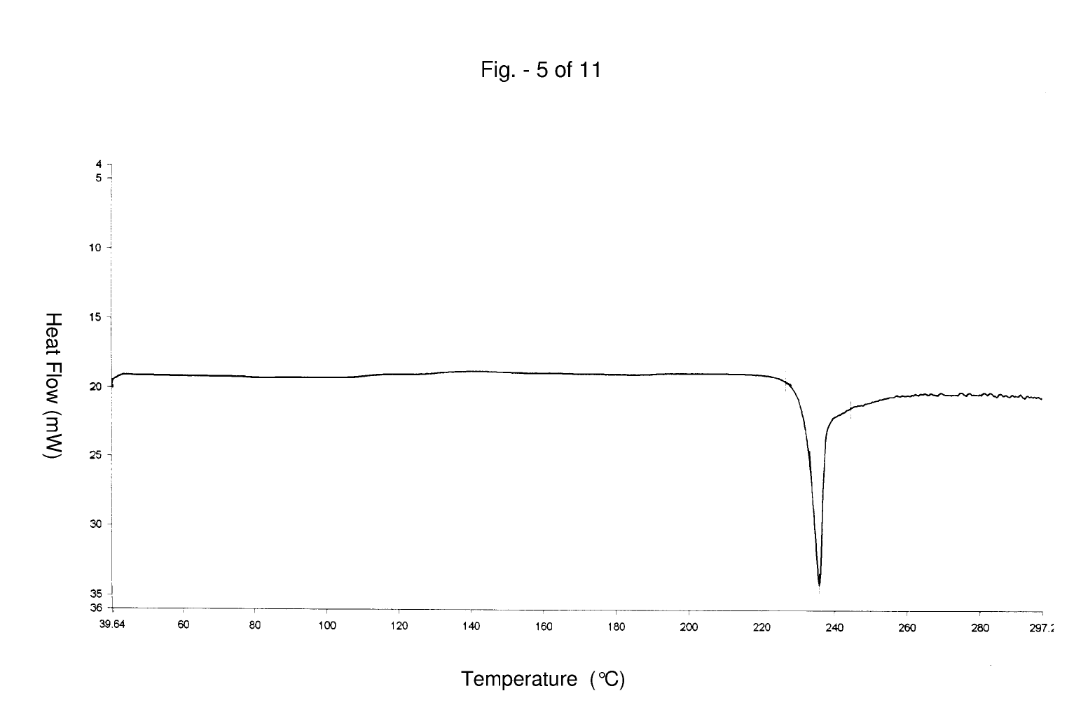

FIG. 7 is an X-ray diffraction pattern of the crystalline Form IV of alfuzosin hydrochloride prepared according to Example 4.

FIG. 8 is a differential scanning calorimetric analysis curve of the crystalline Form IV of alfuzosin hydrochloride prepared according to Example 4.

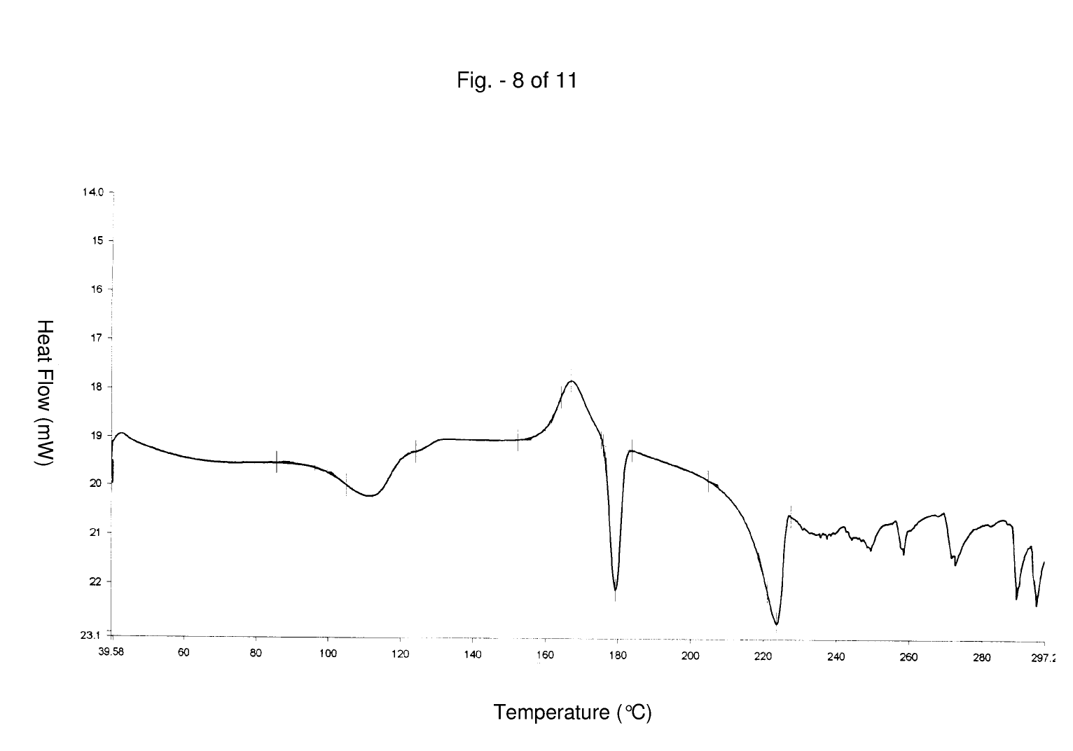

FIG. 9 is a thermogravimetric analysis curve of the crystalline Form IV alfuzosin hydrochloride prepared according to Example 4.

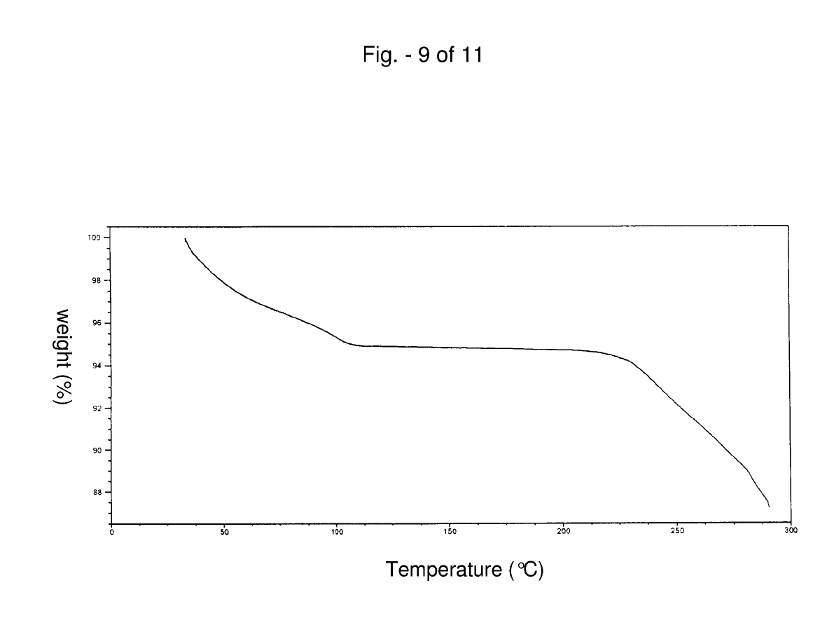

FIG. 10 is an X-ray diffraction pattern of the amorphous form of alfuzosin hydrochloride prepared according to Example 6.

FIG. 11 is an X-ray diffraction pattern of the anhydrous alfuzosin hydrochloride Form I prepared according to Example 7.

### DETAILED DESCRIPTION OF THE INVENTION

According to one aspect, the present invention provides crystalline polymorphic forms of alfuzosin hydrochloride designated as Form II, Form III, and Form IV characterized by their X-ray powder diffraction (XRPD) patterns.

X-ray diffraction patterns are unique for different crystalline forms. Each crystalline form may exhibit a diffraction pattern with a unique set of diffraction peaks that can be expressed in 2θ angles, d-spacing values and relative peak intensities. 2θ diffraction angles and corresponding d-spacing values account for positions of various peaks in the X-ray powder diffraction pattern. d-spacing values can be calculated with observed 2θ angles and copper Kα-1 wavelength radiation using the Bragg equation as is well known to those of skill in the art.

However, slight variations in observed 2θ angles or d-spacing values are expected based on the specific diffractometer employed, preparation techniques and/or other experimental variations. Moreover, the heights of individual peaks can vary considerably between samples of the same substance, frequently because of sample preparation differences and also more variation is expected for the relative peak intensities especially in the low 2θ angle region where the technique is more sensitive to the particle sizes of the sample. Therefore, when comparing XRPD patterns, the peak locations, and not their relative intensities, will be the better indicator of whether the same substances were present in the samples.

All the X-ray powder diffraction patterns were obtained on a Bruker Axe, D8 Advance Powder X-ray Diffractometer with a Cu K alpha-1 radiation source.

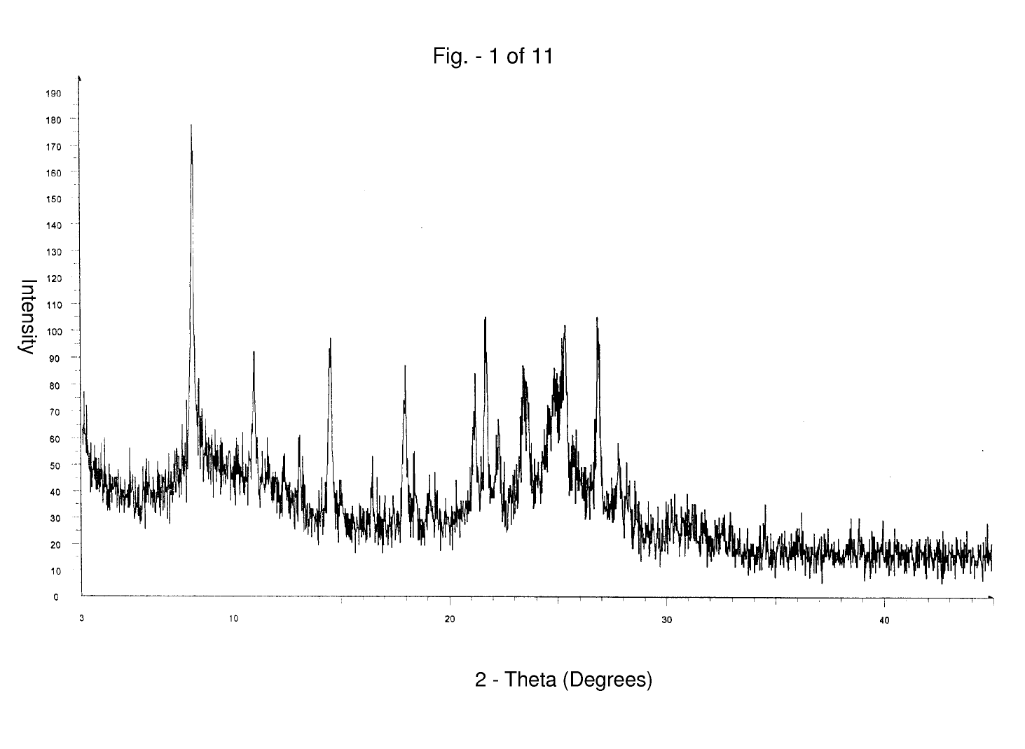

The crystalline Forms II, III, and IV of alfuzosin hydrochloride can be characterized by X-ray diffraction. Crystalline alfuzosin hydrochloride Form II is characterized by its XRPD pattern substantially in accordance with FIG. 1, which pattern comprises significant peaks at about 8.1, 14.4 and 17.9, ±0.2° 2θ. Form II can be further characterized further by the additional XRPD peaks at about 10.9, 21.1, 23.4, 23.5, 24.8, 25.3, and 26.8, ±0.2° 2θ.

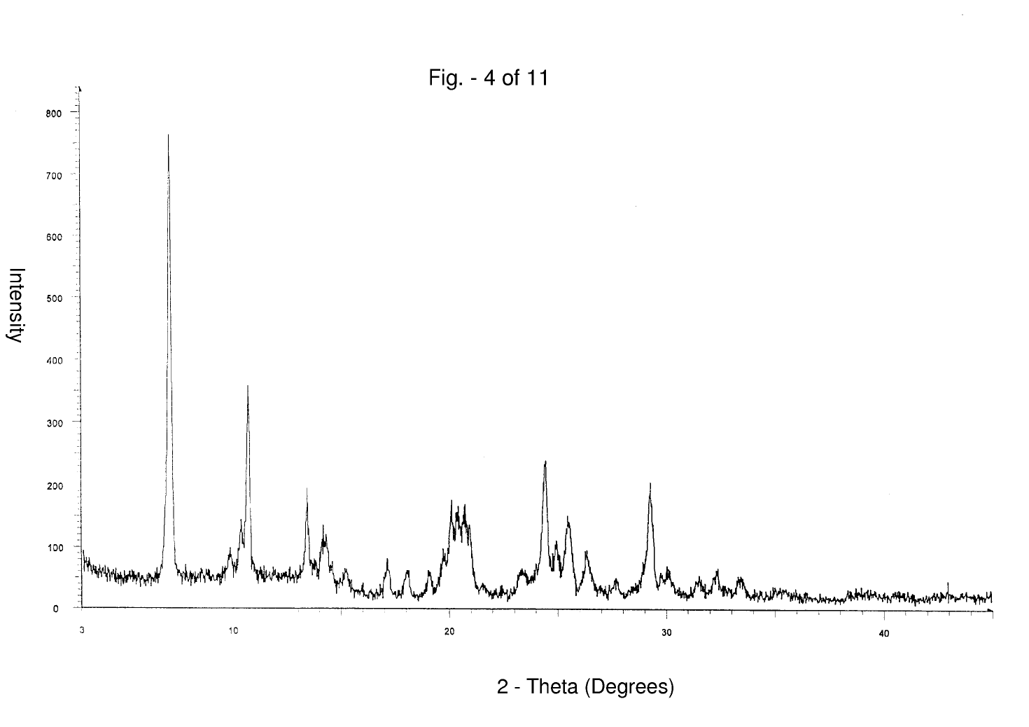

Crystalline alfuzosin hydrochloride Form III is characterized by its XRPD pattern substantially in accordance with FIG. 4, which pattern comprises significant peaks at about 7.4 and 10.6, ±0.2° 2θ. Form III can be further characterized further by the additional XRPD peaks at about 13.3, 20.0, 20.3, 20.6, 24.4, 29.2, ±0.2° 2θ.

Crystalline alfuzosin hydrochloride Form IV is characterized by its XRPD pattern substantially in accordance with FIG. 4, which pattern comprises significant peaks at about 10.0, 18.3 and 23.2, ±0.2° 2θ. Form IV can be further characterized further by the additional XRPD peaks at about 11.0, 12.1, 14.9, 20.1 and 24.6, ±0.2° 2θ.

Thus, by understanding which peaks of a X-ray diffraction pattern of a crystalline form is distinctive over those of other crystalline form of the same compound is particularly useful to determine presence of any form in another form.

In the present invention, the X-ray diffraction patterns of the crystalline Form II shows that the peaks at about 8.1, 14.4 and 17.9, ±0.2° 2θ are particularly useful for identification of the crystalline Form II over the Form III and Form IV, and that the peaks at about 7.4 and 10.6, ±0.2° 2θ of the crystalline Form III are especially distinctive over the peaks of the crystalline Form II and Form IV, and that the peaks at about 10.0, 18.3 and 23.2, ±0.2° 2θ of the crystalline Form IV are especially distinctive over the peaks of the crystalline Form II and Form III.

According to one more aspect, the present invention provides a process for preparing the crystalline Forms II, III, and IV comprising the steps of:

a) reacting alfuzosin free base with hydrochloric acid in a suitable solvent;

b) optionally adding an another solvent; and

c) recovering the desired crystalline form.

The processes of the present invention involves crystallization from a particular solvent, i.e., obtaining a solid material from a solution. One skilled in the art will appreciate that sometimes a specific condition can produce only a particular form and any slight variation in such condition could lead to the formation of another form. For example in the present case selection solvent plays a major role during preparation of crystalline forms of alfuzosin hydrochloride and any slight alternation in the solvents leads to the formation of a different form.

For the preparation of Form II, the suitable solvents that can be used in step a) include C₁-C₄ straight chain alcohols such as methanol, ethanol, and the like, and mixtures thereof.

The second solvent that is optionally used in step b) can be an antisolvent, which includes but is not limited to any solvent or mixture of solvents. Examples include C₃-C₆ branched alcohols such as 2-propanol, 2-butanol and the like, and mixtures thereof.

Optionally the solution can be seeded with small amounts, such as about 0.5 to 1.0% w/w, of crystals of alfuzosin hydrochloride Form II.

For the preparation of Form III, the suitable solvents that can be used in step a) include ketones such as acetone, ethyl methyl ketone, methyl Isobutyl ketone and the like, and mixtures thereof; and chlorohydrocarbons such as dichloromethane, ethylene dichloride, chloroform and the like, and mixtures thereof.

For the preparation of Form IV, the suitable solvents that can be used in step a) include C₆-C₁₀ straight chain, branched or cyclic hydrocarbons such as n-hexane, n-heptane, cyclohexane and the like, and mixtures thereof; and ether solvents such as diethyl ether, diisopropyl ether, methyl tert-butyl ether, tetrahydrofuran and the like, and mixtures thereof.

The suitable reagents for conversion of alfuzosin free base into the hydrochloride in step a) include but are not limited to methanolic hydrochloride, isopropanolic hydrochloride, ethyl acetate hydrochloride, aqueous hydrochloric acid, and the like.

The isolation of the solid of step c) can be carried out by using conventional techniques, such as decantation, centrifugation, gravity filtration, or vacuum filtration.

The drying operation employed in step d) is carried out using any technique, such as fluid bed drying (FBD), spin flash drying, aerial drying, oven drying or other techniques known in the art at temperatures of about 95-105° C. with or without application of vacuum and/or under inert conditions. In an embodiment, the drying is carried out under vacuum at about 100° C.

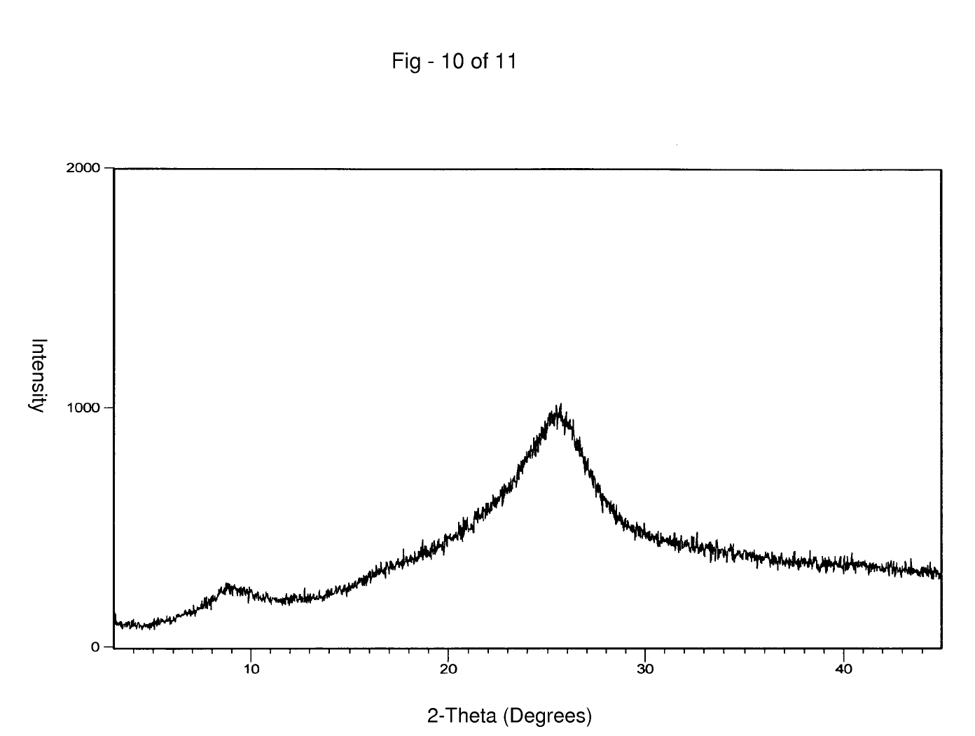

According to one more aspect the present invention also provides an amorphous form of alfuzosin hydrochloride, which is characterized by its X-ray powder diffraction pattern as shown in FIG. 10.

According to an aspect, the present invention provides a process for preparing the amorphous form comprising the steps of:

a) heating alfuzosin hydrochloride comprising water to form a melt; and

b) cooling the liquid of step a) to generate the amorphous form.

Alfuzosin hydrochloride that can be used in the process for preparation of the amorphous form includes hydrates such as mono-, di-, tri-, and tetra-hydrates, and crystalline forms such as Form I, Form II, Form III, and Form IV.

For the preparation of amorphous form, alfuzosin hydrochloride that is used can have a moisture content about 3 to 10%, or 4 to 6%, by weight.

A suitable temperature at which alfuzosin hydrochloride comprising water melts to form a liquid includes about 130 to 135° C., and optionally vacuum is applied.

The liquid thus obtained is allowed to cool to below 45° C., or about 20 to 25° C., to afford the amorphous form of alfuzosin hydrochloride The obtained amorphous form can be further dried at a suitable temperature and optionally by applying vacuum until the solid melts using any technique, such as oven drying, tray drying, rotational drying (such as the Buchi Rotavapor), spray drying, freeze-drying, fluid bed drying, flash drying, spin flash drying and agitated thin film drying, and the like.

The moisture content of the crystalline forms can be characterized using the Karl Fischer technique, such as by preparing a solution of 1 g of sample in 40 ml of methanol and than titrating with Karl Fischer reagent. Thus, crystalline Form II of alfuzosin hydrochloride has a moisture content ranging from about 4 to 6% by weight. Similarly, Form III has a moisture content ranging from about 1 to 3% by weight and Form IV has a moisture content ranging from about 6.5 to 8.5% by weight. The amorphous form of alfuzosin hydrochloride has a moisture about 10% by weight.

In turn, the dehydration behavior of the crystal water of the said forms was measured by using thermogravemetric analysis by using thermogravemetric apparatus (Q 500 TA model). Thus a typical crystalline Form II has a water loss of about 6.6% by weight, a typical crystalline Form III has a water loss of about 1.4% by weight, and a typical Form IV has a water loss of about 5.1% by weight.

Yet in another aspect, the present invention provides a process for conversion of alfuzosin hydrochloride Form II to anhydrous alfuzosin hydrochloride Form I, which comprises heating the alfuzosin hydrochloride Form II at a suitable temperature.

The heating of alfuzosin hydrochloride Form II is carried out using any technique, such as fluid bed drying (FBD), spin flash drying, aerial drying, oven drying or other techniques known in the art, at temperatures about 145-150° C. with or without application of vacuum and/or under inert conditions.

Further in another aspect, the present invention provides a pharmaceutical composition containing, separately or as a mixture, the crystalline Forms II, III, and IV, and the amorphous form, of alfuzosin hydrochloride and one or more pharmaceutically acceptable excipients.

The pharmaceutical composition may be prepared by uniformly admixing the active ingredient with liquid or solid carriers and then formulating product into the desired form. The pharmaceutical compositions may be in the form of suspensions, solutions, elixirs, aerosols, or solid dosage forms. Because of their ease of administration, tablets and capsules represent the most advantageous oral dosage unit form, in which case solid pharmaceutical carriers are employed.

Such pharmaceutical composition may be administered to a mammalian patient in a dosage form. The said compositions can be administered to human or animal orally, parenterally, rectally or by other routes of administration.

Solid dosage forms for oral administration include tablets, capsules, pills, powders, granules, pellets or spheres comprising active substance, pellets or spheres coated with active substance, coated beads, coated microparticles, coated granules and the like.

The pellets or “cores” that can be used include but are not limited to: water-soluble cores such as sugar spheres, lactose, and the like; and water-insoluble cores such as microcrystalline cellulose, silicon dioxide, calcium carbonate, dicalcium phosphate anhydrous, dicalcium phosphate monohydrate, tribasic calcium phosphate, magnesium carbonate, magnesium oxide, and the like.

The solid dosage forms can be prepared by mixing the crystalline Forms II, III and IV and the amorphous form of alfuzosin hydrochloride separately or as a mixture, with pharmaceutically acceptable excipients like diluents, binders, glidants, disintegrants, pH adjusting agents, lubricants, colorants, flavoring agents, surfactants, anti-tack agents, opacifying agents and the like. The compositions may be immediate release or modified release like controlled release or delayed release forms where the release of the active substance can be modified by use of suitable release modifying excipients.

The pharmaceutical compositions of present invention can contain one or more diluents added to increase the tablet or capsule mass making it easy to formulate and handle. Common diluents include, but are not limited to microcrystalline cellulose, powdered cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, and the like.

Binders can also be included in the pharmaceutical compositions of the present invention to help hold granules or tablets together. Some examples include but are not limited to acacia, alginic acid, sodium alginate, gelatin, guar gum, starch, pregelatinized starch, polyvinyl pyrollidone, carboxymethyl cellulose, methyl cellulose, ethyl cellulose, hydroxyethyl cellulose, hydroxypropyl cellulose, hydroxypropyl methyl cellulose, liquid glucose, magnesium aluminium silicate, dextrin, maltodextrin and polymethacrylates.

Lubricants such as talc, stearic acid, magnesium stearate, calcium stearate, sodium stearyl fumarate, colloidal silicon dioxide, glyceryl monostearate and the like can be included in the compositions of present invention.

Useful pH adjusting agents include but are not limited to: alkalizers like sodium carbonate, potassium carbonate, magnesium carbonate, calcium carbonate, sodium hydroxide, magnesium hydroxide, aluminium magnesium hydroxide, magnesium metasilicate aluminate, magnesium silicate aluminate, sodium citrate, potassium citrate, sodium acetate, potassium acetate, organic bases like TRIS (tris(hydroxymethyl)amino methane), arginine, tromethamine and meglumine; and acidifiers like ascorbic acid, citric acid, tartaric acid, fumaric acid, acetic acid, malic acid, maleic acid, formic acid, propionic acid and hydrochloric acid.

Useful wetting agents include but are not limited to gelatin, casein, lecithin, gum acacia, cholesterol, tragacanth, stearic acid, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., TWEEN™), polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxy propylcellulose, hydroxypropylmethylcellulose phthlate, magnesium aluminum silicate, triethanolamine, polyvinyl alcohol and polyvinylpyrrolidone (PVP), poloxamers such as PLURONIC™ F68, F127, and F108 which are block copolymers of ethylene oxide and propylene oxide and polyxamines such as TETRONIC™ 908.

The compositions in the form of tablets or capsules can further include a disintegrant to accelerate disintegration of the said tablet or capsule. Useful disintegrants include but are not limited to alginic acid, carboxymethyl cellulose calcium, carboxymethyl cellulose sodium, colloidal silicon dioxide, crospovidone, guar gum, magnesium aluminium silicate, microcrystalline cellulose, powdered cellulose, polacrillin potassium, sodium alginate, sodium starch glycolate, starch and pregelatinized starch.

The compositions can be prepared using processes known to one skilled in the art, such as direct compression, dry granulation, compaction granulation or wet granulation.

The compositions can be further coated. The coating may be seal coating, film coating, subcoating, barrier coating, polishing coating, compression coating, fast disintegrating coating, enzyme degradable coating, sugar coating, release-modifying coating like polymeric or enteric coat, specialized coatings like bioadhesive coatings, and such the like, or combinations thereof. Coating can be achieved by methods such as by using fluidized bed equipment, perforated pans, a regular pharmaceutical pan, compression coating, continuous or short spray methods, by drenching or such other methods known to one skilled in the art.

Further, multiple coatings can be applied to achieve desired results. The active substance can be present in the core or coat(s) or both. The cores in the form of pellets, granules or tablets may be coated with active substance along with pharmaceutically acceptable excipient(s) like alkalizer and the cores can be optionally coated with release modifying or specialized coating. There can be a separate coating of alkalizer or it can be present in any of the outer coating layers. The coatings can comprise other excipients like plasticizers such as triethyl citrate, acetyl triethyl citrate, acetyl tributyl citrate, acetylated monoglycerides, glycerin, triacetin, propylene glycol, phalate esters, castor oil, sorbitol and dibutyl sebacate; anti tackiness agents like talc and glyceryl monostearate; pigments like titanium dioxide or ferric oxides, polishing agents like glyceryl monostearate, carnauba wax, candellila wax and the like.

The solvents that may be used for preparation of coating composition may be organic or inorganic, including water, or mixtures thereof. Organic solvents like methanol, ethanol, acetone, dichloromethane, isopropanol and the like can be used in the present invention.

The excipients that may be used to modify the release of active substance in the present invention can be hydrophilic or hydrophobic or combinations of both. The hydrophobic release modifying agents can include but are not limited to ethyl cellulose, cellulose acetate, cellulose propionate, cellulose acetate propionate, cellulose acetate butyrate, cellulose triacetate, methacrylic acid copolymer type A, B and C, polymeric acrylate derivatives, pluronic block copolymers, polyvinyl acetate, waxes like beeswax, carnauba wax, microcrystalline wax and ozokerite, fatty alcohols like cetostearyl alcohol, stearyl alcohol, cetyl alcohol and myristyl alcohol, fatty acid esters like glyceryl monostearate, glycerol distearate, glycerol monooleate, acetylated monglyceride, tristearin, tripalmitin, palmitostearate, glyceryl behenate and hydrogenated castor oil.

The hydrophilic release modifying agents that can be used include but are not limited to hydroxymethyl cellulose, hydroxyethyl cellulose, hydroxypropyl cellulose, hydroxybutyl cellulose, hydroxyethyl methyl cellulose, hydroxypropyl methyl cellulose, carboxymethylcellulose, sodium carboxymethylcellulose, carboxymethyl ethyl cellulose, carboxyalkylcellulose esters, natural, semisynthetic or synthetic polysaccharides such as alginic acid, alkali metal and ammonium salts, carrageenans, galactomannans, tragacanth, agar-agar, gum arabic, guar gum, xanthan gum, pectins like sodium carboxymethylamylopectin, chitosan, polyfructans, inulin, polyacrylic acids, polymethacrylic acids, methacrylate copolymers, polyvinyl alcohol; polyvinylpyrrolidone, copolymers of polyvinylpyrrolidone with vinyl acetate, combinations of polyvinyl alcohol and polyvinylpyrrolidone, polyalkylene oxides such as polyethylene oxide and polypropylene oxide and copolymers of ethylene oxide and propylene oxide.

Useful enteric excipients include but are not limited to methacrylic acid copolymers type A, B and C, hydroxypropyl methylcellulose phthalate, hydroxypropyl methylcellulose acetate succinate, cellulose acetyl phthalate, cellulose diacetyl phthalate, cellulose triacetyl phthalate, cellulose acetate phthalate, polyvinyl acetate phthalate, sodium cellulose acetate phthalate, cellulose ester phthalate, cellulose ether phthalate, methylcellulose phthalate, cellulose ester-ether phthalate, hydroxypropyl cellulose phthalate, alkali salts of cellulose acetate phthalate, alkaline earth salts of cellulose acetate phthalate, calcium salts of cellulose acetate phthalate, ammonium salts of hydroxypropyl methylcellulose phthalate, cellulose acetate hexahydrophthalate, hydroxypropyl methylcellulose hexahydrophthalate, polyvinyl acetate phthalate diethyl phthalate, dibutyl phthalate, dialkyl phthalate, cellulose acetate trimelliate, shellac, zein and mixtures thereof.

Certain specific aspects of the present invention will be explained in more detail with reference to the following examples, which are provided by way of illustration only and should not be construed as limiting the scope of the invention in any manner.

### EXAMPLE 1

Preparation of Alfuzosin Hydrochloride Form II

10 g of alfuzosin free base and 70.5 ml of methanol was charged in a 4 neck round bottom flask. 9.5 ml of 12% hydrochloric acid in methanol was added to the suspension at about 34° C. to obtain clear solution. 0.06 g of alfuzosin hydrochloride Form II seed crystals was charged to the solution. Solid separation was observed upon cooling to about 12° C. 100 ml of isopropyl alcohol was added slowly to the suspension and stirred for a period of about 90 minutes at same temperature. Filtered the solid under closed nitrogen atmosphere and washed with 10 ml of isopropyl alcohol. Dried the solid at 60° C. for 3 hours under reduced pressure, then at 110° C. for about 12 hours under reduced pressure to yield 10.8 g of alfuzosin hydrochloride Form II having the XRPD pattern shown in FIG. 1.

Alfuzosin hydrochloride Form II thus obtained has a moisture content of about 4.9% by weight, and has a melting peak at about 235° C.

### EXAMPLE 2

Preparation of Alfuzosin Hydrochloride Form III

5 g of alfuzosin free base was charged into a 4 neck round bottom flask containing 50 ml of acetone. 3.2 ml of 12% hydrochloric acid in isopropanol was added to the suspension at about 30° C. and stirred for 30 minutes at the same temperature. Filtered the solid and dried at 100° C., under reduced pressure, for about 12 hours to yield 5.1 g of alfuzosin hydrochloride Form III.

### EXAMPLE 3

Preparation of Alfuzosin Hydrochloride Form III

5 g of alfuzosin free base was charged into a 4 neck round bottom flask containing 50 ml of dichloromethane. 3.6 ml of 12% hydrochloric acid in isopropanol was added to the suspension at about 30° C. and stirred for 30 minutes at same temperature. Filtered the solid and dried at 100° C., under reduced pressure, for about 12 hours to yield 4.1 g of alfuzosin hydrochloride Form III having the XRPD pattern shown in FIG. 4.

Alfuzosin hydrochloride Form III thus obtained has a moisture content of about 2.7% by weight, and has a melting peak at about 236° C.

### EXAMPLE 4

Preparation of Alfuzosin Hydrochloride Form IV

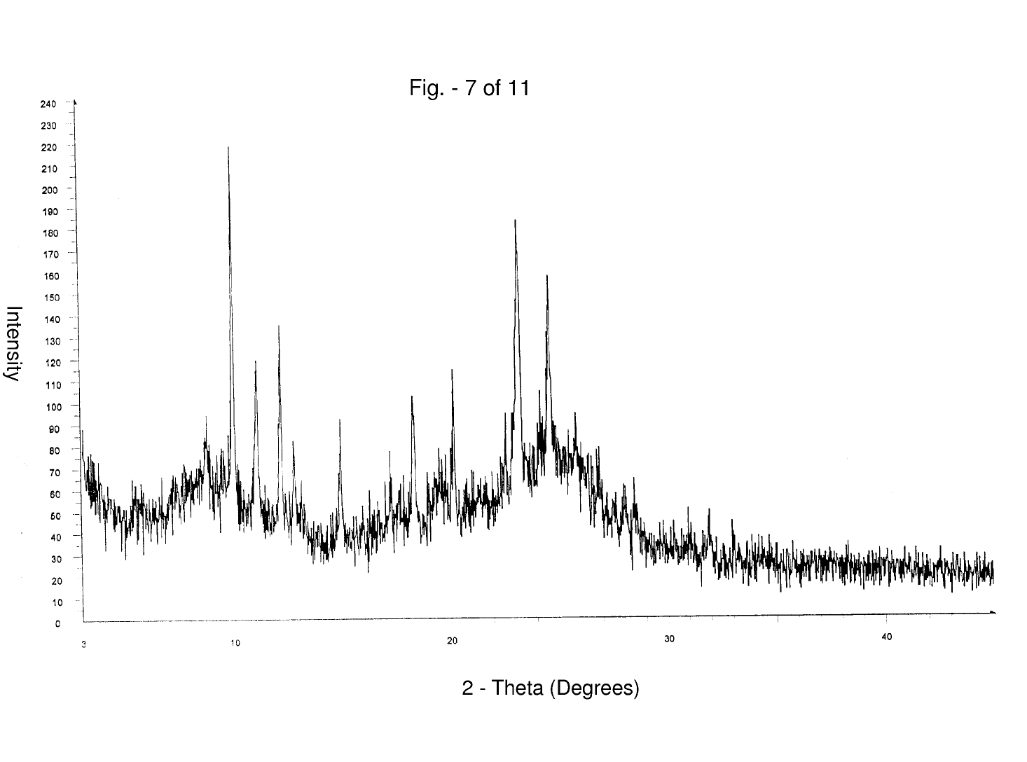

5 g of alfuzosin free base was charged into a 4 neck round bottom flask containing 50 ml of cyclohexane. 3 ml of 12% hydrochloric acid in isopropanol was added to the suspension at about 33° C. and stirred for 30 minutes at same temperature. Filtered the solid and dried at 100° C., under reduced pressure, for about 12 hours to yield 5.2 g of alfuzosin hydrochloride Form IV having the XRPD pattern shown in FIG. 7.

Alfuzosin hydrochloride Form IV thus obtained has a moisture content of about 7.8% by weight, and has a melting peak at about 179° C.

### EXAMPLE 5

Preparation of Alfuzosin Hydrochloride Form IV

5 g of alfuzosin free base was charged into a 4 neck round bottom flask containing 50 ml of methyl t-butyl ether. 3 ml of 12% hydrochloric acid in isopropanol was added to the suspension at about 26° C. and stirred for 30 minutes at the same temperature. Filtered the solid and dried at 100° C., under reduced pressure, for about 10 hours to yield 5.4 g of alfuzosin hydrochloride Form IV.

### EXAMPLE 6

Preparation of Alfuzosin Hydrochloride Amorphous Form

20 g of alfuzosin free base was charged into a 4 neck round bottom flask containing 82 ml of methanol. 18 ml of 12% hydrochloric acid in methanol was added to the suspension at about 30° C. and then cooled to 12° C. Stirred at same temperature for 60 minutes. 200 ml of isopropanol was added slowly to the mass for 60 minutes and stirred for about 90 minutes at 12° C. Filtered the solid and washed with methanol.

The wet solid thus obtained (having a moisture content about 5% by weight) was charged in a clean dry round bottom Buchi Rotavapor flask and heated to about 135° C. under vacuum. The solid was melted and again solidified during the course of maintenance. The flask was then cooled to about 28° C. and the solid collected to get 21.6 g of alfuzosin hydrochloride amorphous form having the XRPD pattern shown in FIG. 10.

Amorphous alfuzosin hydrochloride thus obtained has a moisture content of about 10% by weight, and has a melting peak at about 233° C.

### EXAMPLE 7

Conversion of Alfuzosin Hydrochloride Form II into Anhydrous Alfuzosin Hydrochloride Form I

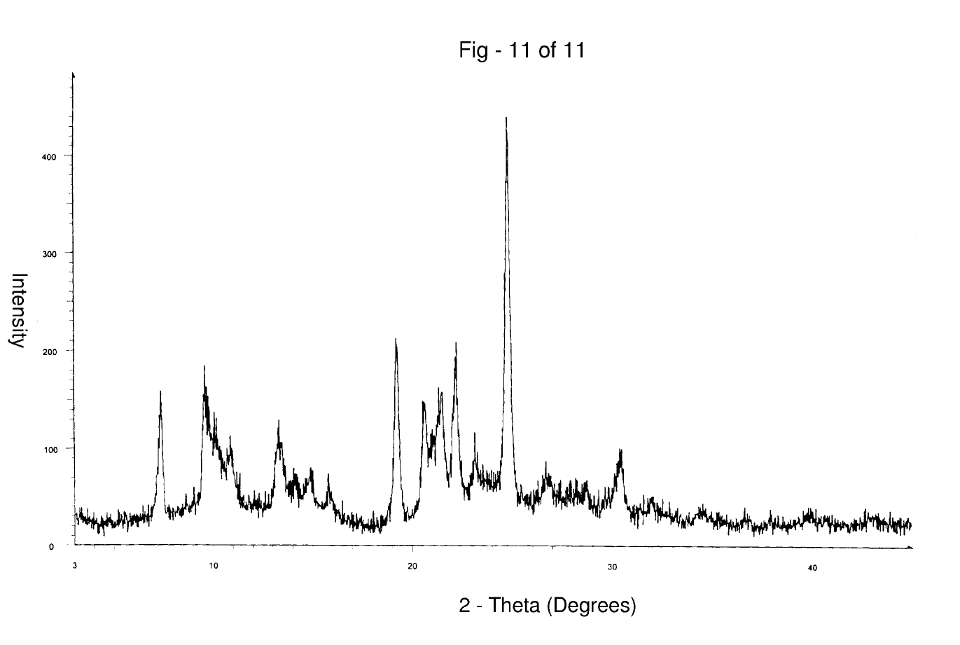

20 g of alfuzosin hydrochloride Form II (prepared in Example 1) was charged into a clean, dry round bottom Buchi Rotavapor flask and heated to about 150° C. Dried the solid under vacuum at the same temperature under reduced pressure for about 8 hours to yield 19 g of anhydrous alfuzosin hydrochloride Form I having the X-ray diffraction pattern of FIG. 11.

1. Alfuzosin hydrochloride having crystalline Form II.

2. The alfuzosin hydrochloride of claim 1, having an X-ray diffraction pattern substantially in accordance with the pattern of FIG. 1.

3. A process for preparing the alfuzosin hydrochloride of claim 1, comprising reacting alfuzosin free base with hydrochloric acid in a C₁-C₄ straight chain alcohol solvent, and adding a C₃-C₆ branched alcohol.

4. The process of claim 3, wherein a C₁-C₄ straight chain alcohol comprises methanol.

5. The process of claim 3, wherein a C₃-C₆ branched alcohol comprises isopropanol.

6. The process of claim 3, wherein seed crystals of alfuzosin hydrochloride Form II are added.

7. Alfuzosin hydrochloride having crystalline Form III.

8. The alfuzosin hydrochloride of claim 7, having an X-ray diffraction pattern substantially in accordance with the pattern of FIG. 4.

9. A process for preparing the alfuzosin hydrochloride of claim 7, comprising reacting alfuzosin free base with hydrochloric acid in a ketone or chlorohydrocarbon solvent.

10. The process of claim 9, wherein a ketone comprises acetone.

11. The process of claim 9, wherein a chlorohydrocarbon comprises dichloromethane.

12. Alfuzosin hydrochloride having crystalline Form IV.

13. The alfuzosin hydrochloride of claim 12, having an X-ray diffraction pattern substantially in accordance with the pattern of FIG. 7.

14. A process for preparing the alfuzosin hydrochloride of claim 12, comprising reacting alfuzosin free base with hydrochloric acid in a C₆-C₁₀ straight chain, branched or cyclic hydrocarbon solvent, or an ether solvent.

15. The process of claim 14, wherein a C₆-C₁₀ straight chain, branched or cyclic hydrocarbon comprises cyclohexane.

16. The process of claim 14, wherein an ether comprises methyl t-butyl ether.

17. Amorphous alfuzosin hydrochloride.

18. A process for preparing the amorphous alfuzosin hydrochloride of claim 17, comprising melting an alfuzosin hydrochloride having a moisture content about 3 to 10 percent by weight, and cooling to temperatures below about 45° C.

19. The process of claim 18, wherein an alfuzosin hydrochloride has a moisture content about 4 to 6 percent by weight.

20. A process for preparing anhydrous alfuzosin hydrochloride having crystalline Form I, comprising heating alfuzosin hydrochloride having crystalline Form II to temperatures about 145-150° C.
